(19) United States
(12) Patent Application Publication
(10) Pub. No.: US 2026/0194455 A1
(43) Pub. Date: Jul. 9, 2026

(54) DEVICE FOR MULTIPLEXED OPTICAL BIOSENSING

(71) Applicant: SiPhox, Inc., Burlington, MA (US)

(72) Inventors: Kyle Preston, Guilford, CT (US); Ebrahim Aljohani, Somerville, MA (US); Sarat Gundavarapu, Cambridge, MA (US); Michael Dubrovsky, Cambridge, MA (US); Diedrik Vermeulen, Arlington, MA (US)

(73) Assignee: SiPhox, Inc., Burlington, MA (US)

(21) Appl. No.: 19/252,055

(22) Filed: Jun. 27, 2025

Related U.S. Application Data
(60) Provisional application No. 63/741,611, filed on Jan. 3, 2025.

(51) Int. Cl. G01N 21/25; G01N 33/543

(52) U.S. Cl. CPC G01N 21/255; G01N 33/54373; G01N 2201/08

(57) ABSTRACT

In an aspect, a device for multiplexed optical biosensing includes at least an optical waveguide, at least a light source optically coupled to the input thereof, a plurality of optical signal modulators on the waveguide, each fluidically connected to a fluidic channel containing a biological analyte and each providing modified modifies the input signal based on the at least a biological analyte, and each modified signal has a distinct optical characteristic, and a receiver module optically coupled to the output of each optical waveguide and including at least a light sensor optically coupled to the output of the optical waveguide and configured to detect the plurality of modified signals and circuitry configured to record the plurality of detected modified signals, separate them, and detect analytes using them.

### CROSS-REFERENCE TO RELATED APPLICATIONS

This application claims the benefit of priority of U.S. Provisional Patent Application Ser. No. 63/741,611, filed on Jan. 3, 2025, and titled “MULTIPLEXED PHOTONIC BIOSENSORS,” which is incorporated by reference herein in its entirety.

### FIELD OF THE INVENTION

The present invention generally relates to the field of optical analysis of biochemical assays. In particular, the present invention is directed to a device for multiplexed optical biosensing.

### BACKGROUND

Biosensors utilizing photonic integrated circuits (PICs) have shown significant promise due to their high sensitivity, compactness, and scalability. However, traditional PIC-based sensors face challenges in increasing sensing capacity (plexity) due to limitations in physical input 108a-n/output (I/O) ports, leading to complex and costly designs.

### SUMMARY OF THE DISCLOSURE

In an aspect, a device for multiplexed optical biosensing includes at least an optical waveguide, each optical waveguide of the at least an optical waveguide having an input and an output, at least a light source optically coupled to the input of each optical waveguide, wherein the at least a light source is configured to transmit an input signal into the optical waveguide, a plurality of optical signal modulators optically coupled to each optical waveguide, wherein each optical signal modulator of the plurality of optical signal modulators is fluidically connected to a fluidic channel containing at least a biological analyte and the plurality of optical signal modulators is configured to produce a plurality of modified signals, wherein each modified signal of the plurality of modified signals is generated by an optical signal modulator of the plurality of optical signal modulators by modifying the input signal based on the at least a biological analyte, and each modified signal of the plurality of modified signals has a distinct optical characteristic from all other modified signals of the plurality of modified signals, and a receiver module optically coupled to the output of each optical waveguide, the receiver module further including at least a light sensor optically coupled to the output of the optical waveguide and configured to detect the plurality of modified signals and circuitry configured to record the plurality of detected modified signals, wherein the receiver module is configured to detect each modified signal of the plurality of modified signals based on its distinct optical characteristic and determine presence of each of the at least a biological analyte based on a respective detected modified signal.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a device for multiplexed photonic biosensors;

FIG. 2 illustrates an example biosensor chip containing up to 2 biosensors per optical output channel;

FIG. 3 illustrates an example biosensor chip containing up to 4 biosensors per optical output channel;

FIGS. 4A-C illustrate exemplary embodiments of optical signal modulators;

FIG. 5A illustrates an exemplary embodiment of transmission spectra of two multiplexed resonators;

FIG. 5B illustrates an exemplary embodiment of transmission spectra of four multiplexed resonators;

FIG. 6 is a block diagram illustrating an exemplary embodiment of a machine-learning module;

FIG. 7 is a schematic diagram illustrating an exemplary embodiment of a neural network;

FIG. 8 is a schematic diagram illustrating an exemplary embodiment of a node of a neural network; and

FIG. 9 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

The present disclosure generally relates to the design and methods of multi-photonic biosensors for multiplexed, label-free or labeled biosensing, which may be used, without limitation, in point-of-care (POC) or at-home diagnostic systems. Generally, the present disclosure discusses designs and methods for multiplexing multiple biosensors per optical channel on a biosensing chip. It is desirable to increase the number of biosensors on a single chip in order to increase the number of multiplexed biomarker measurements obtained from a single sample, such as a blood sample, nasal sample, urine sample, or other biological sample. It is also desirable to minimize the cost and complexity of the chip and the system. Embodiments disclosed herein may involve multiplexing more than one biosensor per optical output channel. Types of biosensors may include devices such as resonators 128, interferometers 132, photonic crystals, or Fabry-Perot cavities, among others.

In some embodiments, two or more resonators 128 multiplexed together on a common optical bus waveguide may be designed to have different optical properties which can be used to distinguish between the multiplexed resonators 128. These properties may include a free spectral range (FSR), extinction ratio, and/or linewidth of the resonances in a transmission spectrum of the devices. Resonator 128 devices may include devices such as microring resonators, racetrack resonators, photonic crystal resonators, and/or Fabry-Perot resonators.

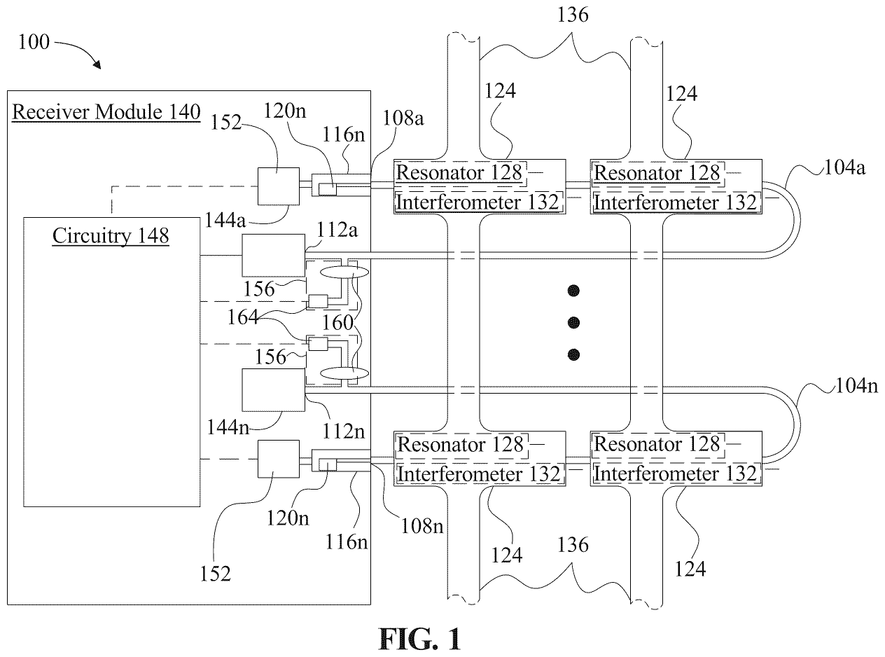

Referring now to FIG. 1, an exemplary embodiment of a device 100 for multiplexed optical biosensing. Device may be included in one or more housings (not shown); for instance, and without limitation, device may include a reader device which is optically or otherwise coupled to a separable biosensor device, which may be disposable, as described in further detail below. Device 100 includes at least an optical waveguide 104a-n, each optical waveguide of the at least an optical waveguide 104a-n having an input 108a-n and an output 112a-n. As used in this disclosure, a “waveguide” is an element configured for propagation of electromagnetic waves. In some cases, a waveguide may be configured to propagate an electromagnetic (EM) wave by any of total internal reflection, attenuated total internal reflection, and/or frustrated internal reflection. In some cases, a waveguide may be configured to propagate an EM wave through reflection, transmission, and/or scattering. In some cases, a waveguide may be configured to propagate electromagnetic radiation (EMR) through surface plasmons such as, for example and without limitation, through surface plasmon resonance. Surface plasmon resonance (SPR) may include resonant oscillation of conduction electrons, for instance, and without limitation, at an interface between negative and positive permittivity material stimulated by incident light. SPR may alternatively or additionally be used to measure adsorption of material onto planar metal, such as, and without limitation, gold or silver, surfaces or onto a surface of metal nanoparticles, for instance, and without limitation, if metal nanoparticles are used as detectable marker, as described above.

As used in this disclosure, an “evanescent wave” (also referred to in this disclosure as an “evanescent field”) may result from EM wave propagation within waveguide. An evanescent wave may exhibit a rapidly decaying (or vanishing) field amplitude in a certain spatial direction, for example, orthogonal to surface of waveguides. In some cases, an evanescent wave may not contribute to energy transport in a spatial direction such as a direction in which evanescent wave exhibits a rapidly decaying or vanishing field amplitude, although in some cases a Poynting vector (averaged over one oscillation cycle) may have non-zero components in other directions. Evanescent wave may be used to detect binding, such as and without limitation, antigen, and/or antibody binding. In some cases, a light signal detected by sensor may indicate presence and/or absence of a particular antigen. For example, and without limitation, an attenuated light signal may indicate that antigen having a highly absorbent detectable marker is proximal to s surface of a waveguide, as the attenuated light signal may result from evanescent wave coupling, for instance via absorption, into detectable marker. As an evanescent wave “vanishes” along a certain direction, its field amplitude, and therefore ability to be used for sensing, may diminish drastically as distance away from surface increases. For example, depending upon parameters, such as index of refraction, light wavelength, light coupling angle, to name a few, an evanescent wave may practically propagate less than 1 μm from surface, less than 100 nm from surface, or even less than 10 nm from surface.

Still referring to FIG. 1, device 100 includes at least a light source 116a-n. Light source 116a-n may be configured to emit a light, which may be emitted into and/or at waveguide; in other words, light source 116a-n may be optically coupled to at least a waveguide. As used in this disclosure, a “light source 116a-n” is any device configured to emit a light. Light source 116a-n may include a coherent light source 116a-n and/or an incoherent light source 116a-n. Nonlimiting exemplary light source 116a-n include lasers, light emitting diodes (LEDs), organic LEDs (OLEDS), light emitting capacitors, incandescent lamps, fluorescent lamps, and the like. In various embodiments, device 100 may include a plurality of light source 116a-n. In some cases, light source 116a-n may include a coherent light source 116a-n, which is configured to emit coherent light, such as without limitation a laser. In some cases, light source 116a-n may include a non-coherent light source 116a-n configured to emit non-coherent light, for example and without limitation a light emitting diode (LED). In some cases, light source 116a-n may emit a light having substantially one wavelength. In some cases, light source 116a-n may emit a light having a wavelength range. Light may have a wavelength in an ultraviolet range, a visible range, a near-infrared range, a mid-infrared range, and/or a far-infrared range. For example, in some cases light may have a wavelength within a range from about 100 nm to about 20 micrometers. In some cases, light may have a wavelength within a range of about 400 nm to about 2,500 nm. Light source 116a-n may include, one or more diode lasers, which may be fabricated, without limitation, as an element of an integrated circuit; diode lasers may include, without limitation, a Fabry Perot cavity laser, which may have multiple modes permitting outputting light of multiple wavelengths, a quantum dot and/or quantum well-based Fabry Perot cavity laser, an external cavity laser, a mode-locked laser such as a gain-absorber system, configured to output light of multiple wavelengths, a distributed feedback (DFB) laser, a distributed Bragg reflector (DBR) laser, an optical frequency comb, and/or a vertical cavity surface emitting laser. Light source 116a-n may additionally or alternatively include a light-emitting diode (LED), an organic LED (OLED) and/or any other light emitter. In some cases, first optical output 112a-n and second optical output 112a-n may be combined along a shared optical path. Light may be coupled into waveguide and propagate within waveguide, for example and without limitation using total internal reflection. In some cases, light may exit waveguide and be detected by a sensor, for instance and without limitation as described in further detail below, upon exiting waveguide. Waveguide may include any structure that may guide waves, such as electromagnetic waves or sound waves, by restricting at least a direction of propagation of the waves. Waves in open space may propagate in multiple directions, for instance in a spherical distribution from a point source. A waveguide may confine a wave to propagate in a restricted sent of directions, such as propagation in one dimension, one direction, or the like, so that the wave does not lose power, for instance to the inverse-square law, while propagating, and/or so that the wave is directed to a desired destination such as a sensor, light detector, or the like. In an embodiment, a waveguide may exploit total reflection at walls, confining waves to the interior of a waveguide. For example, and without limitation, waveguide may include a hollow conductive metal pipe used to carry high frequency radio waves, such as microwaves. Waveguide may include optical waveguides that, when used at optical frequencies, are dielectric waveguides whereby a structure with a dielectric material with high permittivity and thus a high index of refraction may be surrounded by a material with a material with lower permittivity. Such a waveguide may include an optical fiber, such as used in fiberoptic devices or conduits. Optical fiber may include a flexible transparent fiber made from silica or plastic that includes a core surrounded by a transparent cladding material with a lower index of refraction. Light may be kept in the core of the optical fiber by the phenomenon of total internal reflection which may cause the fiber to act as a waveguide. Fibers may include both single-mode and multi-mode fibers. Acting as a waveguide, fibers may support one or more fined transverse modes by which light can propagate along the fiber. Waveguides may be made from materials such as silica, fluorozirconate, fluoroaluminate, chalcogenide glass, sapphire, fluoride, and/or plastic.

Continuing to refer to FIG. 1, light source 116a-n may output one or more wavelengths; for instance, light source 116a-n may include a coherent light source 116a-n such as a laser and/or a source such as a monochromatic LED, which produces only one wavelength or a very narrow band of wavelengths. Alternatively or additionally, light source 116a-n may produce a plurality of wavelengths. In some embodiments, light source 116a-n may include a light source 116a-n that emits a simultaneous frequency continuum, where a “simultaneous frequency continuum” is defined as multiple wavelengths or a spectrum of wavelengths emitted simultaneously, such as without limitation a white light source 116a-n. Alternatively or additionally, light source 116a-n may have a temporal frequency continuum. A “temporal frequency continuum,” as used in this disclosure, is a range of frequencies, which may include a continuous range (also known as a “continuous temporal frequency continuum”) that a light source 116a-n may emit at different times, depending on varying parameters of or at the light source 116a-n. As a non-limiting example, light source 116a-n may include a source that emits only one wavelength or a very narrow band of wavelength at any given time, but can be tuned to modify that wavelength; for instance, light source 116a-n may include a tunable laser 120a-n such as without limitation a tunable laser 120a-n. In a non-limiting example, a tunable laser 120a-n may include a tunable diode laser such as a tunable distributed feedback (DFB) laser such as a DFB laser built using Bragg reflectors or other diffraction gratings as terminal reflectors. A tunable laser 120a-n may be tunable, without limitation, by modification of a temperature of an active region of the laser, varying a diode current of the laser, varying adjustment of a grating angle of the laser, modifying a cavity length of the laser, and/or by modifications to ohmic heating of the laser; each of the above parameters may be modified by a circuit, which may be communicatively connected to, incorporated in, and/or operated by a processor or other control communicatively connected to receiver module 140 as described in further detail below. In an embodiment, light source 116a-n may be programmed to sweep through a range of wavelengths, such as from a minimal wavelength in a tunable range of the light source 116a-n to a maximal wavelength thereof or vice-versa; sweep may be simultaneously recorded and/or matched with output 112a-n from waveguide by circuitry as described in further detail below. At least a light source 116a-n is configured to transmit an input signal into the optical waveguide, for instance and without limitation as described above. Input signal may include without limitation any signal formed by emission of light, including a pulse, a pulse train, a gradually increasing and/or decreasing signal, and/or a signal that varies in intensity, frequency, phase, or any other optical property over time according to any function of time.

With continued reference to FIG. 1, device 100 includes a plurality of optical signal modulators 124 optically coupled to each optical waveguide. An “optical signal modulator,” as used in this disclosure, is an optical component that modifies an optical parameter of a light or photonic signal in a manner that indicates presence or absence of a biological or chemical analyte; optical signal modulators 124 may include, without limitation, resonators 128, interferometers 132, photonic crystals, Fabry-Perot cavities, and/or other components as described in further detail below. Coupling of optical signal modulators 124 to waveguides may be performed, without limitation, by placing an optical signal modulator 124 in line with the waveguide and/or making the optical signal modulator 124 a part of the waveguide, by having the optical signal modulator 124 receive and/or interact with an evanescent wave of the waveguide, and/or using a coupler such as without limitation a grating coupler.

Still referring to FIG. 1, plurality of optical signal modulators 124 is configured to produce a plurality of modified signals. A “modified signal,” as used in this disclosure, is a photonic and/or light signal for which one or more optical properties has been changed; for instance, a spectrum of frequencies, an intensity, a phase, a polarization, and/or an arrangement of intensity levels or other attributes across a spectrum or range of frequencies or the like may be modified in a manner that can be detected using components as described in further detail below. Each modified signal of the plurality of modified signals is generated by an optical signal modulator 124 of the plurality of optical signal modulators 124 by modifying the input signal based on the at least a biological analyte. Each modified signal of the plurality of modified signals may have a distinct optical characteristic from all other modified signals of the plurality of modified signals. For instance, and without limitation, distinct optical properties may include a free spectral range (FSR) that does not match an FSR of each other optical signal modulator 124, an extinction ratio that is distinct from an extinction ratio of each other optical signal modulator 124, and/or one or more linewidth of one or more resonances in a transmission spectrum of an optical signal modulator 124 that does not match linewidths of resonances of transition spectra of other optical signal modulators 124. Further examples of particular distinct optical characteristics produced by distinct optical signal modulators 124 will be described in further detail below.

With continued reference to FIG. 1, at least an optical signal modulator 124 may include, without limitation, a resonator 128. As used in this disclosure, a “resonator 128” is an optical component that has at least one resonant frequency; a resonator 128 optically coupled to a waveguide may cause at least a resonant frequency to be amplified in the coupled waveguide or to be attenuated in the waveguide. A resonator 128 may include, without limitation, a ring resonator 128, defined as a set of waveguides in which at least one waveguide is a closed loop coupled to some sort of light input or output. A ring resonator 128 or similar structure may be implemented and/or utilized as described in U.S. Nonprovisional application Ser. No. 17/859,877, filed on Jul. 7, 2022 under attorney docket number 1214-004USU1 and entitled “DEVICES, SYSTEMS, AND METHODS FOR RESPIRATORY DISEASE TESTING,” the entirety of which is incorporated herein by reference. A resonator 128 may include a racetrack resonator 128, defined as a ring resonator 128 and/or microring resonator 128 that has a straight section, creating a shape that can be reminiscent of a track and field or chariot racetrack; in some embodiments, the straight section of the racetrack resonator 128 may run parallel to a waveguide such as at least an optical waveguide 104a-n, creating a relatively long coupling length, which may improve coupling efficiency. Alternatively or additionally, a portion of at least an optical waveguide 104a-n may run parallel to a portion of a resonator 128 regardless of shape or straightness thereof. A resonator 128 may alternatively or additionally include a spiral resonator 128, defined as a ring or loop resonator 128 in which the closed-loop portion is arranged as a spiral; for instance, a spiral in which light travels from an outer point in the spiral into the center on a first path, follows a second path that spirals back out of the center, and connects again to the inward path, forming a loop in a spiral configuration. In an embodiment, a resonator 128's resonant frequency depends on a length of an optical path in the resonator 128 and its effective refractive index; for instance, where a resonator 128 is a ring resonator 128, the resonant frequency may depend on the internal and external radii of the resonator 128, the refractive index of the material of the resonator 128, and the effective refractive index thereof, which may depend in turn on the refractive index of the waveguide material and the refractive index and/or other optical properties of material external to the resonator 128 such as without limitation fluid medium in which the resonator 128 is located and/or substrates deposited thereon. Thus, modification of substrate and/or fluidic materials around a resonator 128 may change a wavelength amplified or attenuated by the resonator 128 in an associated waveguide such as at least an optical waveguide 104a-n, creating a detectable optical signal from material changes on or around the resonator 128.

Still referring to FIG. 1, an optical signal modulator 124 of plurality of signal modulators may include an interferometer 132. An interferometer 132 is an optical device that uses interference between two beams of light to extract information concerning that light. An interferometer 132 may function by causing two beams of light, such as two beams created by splitting a single beam into two paths that reconnect, to interact, generating a pattern of greater or lesser intensity based on interference between light of opposite phases and amplification (doubling) of light having matching phases. As the length of the beams may remain constant, the phase difference between beams at the point of merger may depend on wavelength; thus, a repeating sinusoidal sequence of gradual increase of intensity to an amplitude-doubling same-phase signal and a gradual decrease to an amplitude-canceling null or attenuated opposite-phase signal, known as an “interference pattern,” may occur as an input signal varies from a low end of a frequency range to a high end of the frequency range or vice-versa. This pattern of varying intensity may be observed, without limitation, by recording an intensity of light emitted from the interferometer 132 as a coherent light source 116a-n is swept through a range of frequencies, for instance and without limitation using a tunable laser 120a-n as described above. An interferometer 132's interference pattern may depend on a length of each of the two beams and an effective refractive index of waveguide material or material traversed by the two beams, which may be modified, as described above for resonators 128, by a medium surrounding an interferometer 132 and/or a substrate deposited thereon, such that modifications to either may produce detectable differences in the interference pattern. Interferometers 132 and/or interferometer 132 devices may include, without limitation, devices such as Mach-Zehnder interferometers 132 or Michelson interferometers 132.

Further referring to FIG. 1, each optical signal modulator 124 of the plurality of optical signal modulators 124 is fluidically connected to a fluidic channel 136 containing at least a biological analyte. Device 100 may include a plurality of fluidics channels. In some embodiments, plurality of fluidics channels may include a modulator-specific channel for each optical modulator of the plurality of optical modulators. Plurality of fluidics channels may be configured to deliver one or more solutions and/or suspensions of chemical and/or biochemical material to optical signal modulators 124 of the plurality of optical signal modulators 124. Material delivered to optical signal modulators 124 may include, without limitation, biological and/or chemical analytes, biological and/or chemical samples to be tested for analytes. Material delivered to optical signal modulators 124 may include, without limitation, “marker” particles and/or chemicals such as metal particles, particles or compounds that are fluorescent, particles or compounds that emit photons according to a known emission spectrum, particles and/or compounds. Material may be delivered, without limitation, as described in matters incorporated herein by reference.

Each or any optical signal modulator 124 of plurality of optical signal modulators 124 may have one or more chemical and/or biochemical substrates deposited thereon. Substrate may include materials that bind to analytes, are modified by analytes, and/or are removed from a surface of optical signal modulators 124 by analytes, which may modify a signal passing through waveguides and/or optical signal modulator 124. For instance, and without limitation, one or more antibodies may be detected using a “sandwich” immunoassay, where a “sandwich” immunoassay includes a photonic waveguide (e.g., resonator 128 or interferometer 132) coated with a capture antibody capable of selectively binding to the antigen desired for detection. In some cases, detection of the antibody may be enhanced by conjugating it with a detectable marker, such as a contrast agent (e.g., gold nanoparticles, Horseradish peroxidase, magnetic nanoparticles, and the like). In some cases, detection of antibody may be alternatively or additionally enhanced by conjugation with a fluorescent marker. Additionally or alternatively, antibodies against certain antigens may be detected by first attaching the antigens, e.g., capture antigens, to photonic waveguide (e.g., resonator 128 and/or interferometer 132) surface. In some examples, anti-human secondary antibodies, e.g., detection antibodies, may be used to amplify the signal or to attain specificity to a certain antibody type (e.g., IgG, IgA, IgM, and the like). In some cases, detection antibodies may be conjugated with a detectable marker, such as a contract agent (e.g., gold nanoparticles, Horseradish peroxidase, magnetic nanoparticle, and the like) or fluorescent agent to improve the signal detected by a sensor, for instance and without limitation as discussed in this disclosure. In one or more embodiments, a sensor that is in communication with the at least an optical waveguide 104a-n may be configured to detect, using modified optical signals, a first constituent of the sample and/or a second constituent of the sample, as a function of a signal modification, which may include, without limitation, a first variance in the first optical property and a second variance in the second optical property. In one or more embodiments, device may include a multiplexor, which may be in communication with the at least an optical waveguide, and a sensor, wherein the multiplexor may be configured to multiplex two or more optical waves.

Continuing to refer to FIG. 1, plurality of optical signal modulators may include a first optical signal modulator 124 configured to generate a first modified signal type and a second optical signal modulator 124 configured to generate a second modified signal type, where the first modified signal type is distinct from the second modified signal type. For instance, a first optical signal modulator 124 may include an interferometer 132, and a second optical signal modulator 124 may include a ring resonator 128, which may cause an output from first optical signal modulator 124 to have a different form from an output of second optical signal modulator 124. As a non-limiting example, where one or more resonators 128 and one or more interferometers 132 are multiplexed together on a common optical bus waveguide, such resonators 128 and interferometers 132 may be designed to have different optical properties which can be used to distinguish between the multiplexed devices. Components as described below may be configured to distinguish a broadband sinusoid response of an interferometer 132 from a narrowband response of a resonator 128 using signal processing, as described in further detail below.

Alternatively or additionally, and still referring to FIG. 1, distinct optical characteristic may include a distinct frequency transmission spectrum. As used in this disclosure, a “distinct frequency transmission spectrum” is a frequency transmission spectrum that has at least one frequency band transmitted in a level of intensity that is measurably different from the way that at least one frequency band is transmitted by each other optical signal modulator 124 sharing the same waveguide of the at least an optical waveguide 104a-n; for example, a distinct frequency transmission spectrum may have at least one frequency band that is transmitted in that frequency transmission spectrum that is not transmitted in other frequency spectra in modified optical signals that are transmitted on the same waveguide, a frequency band that is not transmitted in that transmission spectrum that is transmitted by all other optical signal modulators 124 sharing the same waveguide of plurality of waveguides, and/or distinct patterns of amplified and attenuated signals, such as distinct interference patterns, from all other patterns thereof produced by other optical frequency modulators, whether they are of distinct types of optical frequency modulators or not. As a further non-limiting example, each modified signal of the plurality of modified signals includes at least one frequency domain extremum that does not overlap with an extremum of any other modified signal of the plurality of modified signals, where a “frequency domain extremum” is a local or universal minimum or maximum of intensity as a function of frequency, such as an attenuated or nulled signal captured by a resonator 128, an amplified signal, and/or a peak or trough of a sinusoidal or other frequency-variant transmission and/or interference pattern.

As a non-limiting example, and still referring to FIG. 1, in some embodiments, two or more interferometers 132 may be multiplexed together on a common optical bus waveguide, where the interferometers 132 may be designed to have different optical properties which can be used to distinguish between the multiplexed interferometers 132. These properties may include the FSR, extinction ratio, and/or insertion loss of the transmission spectrum through the devices. Interferometer 132 devices could include devices such as Mach-Zehnder interferometers 132 and Michelson interferometers 132.

In some embodiments, and still referring to FIG. 1, an FSR of a biosensor may be more accurately controlled in chip fabrication than an absolute wavelength of a particular resonance. Therefore, multiplexed resonances on a single optical channel may have well-controlled free spectral ranges but an uncontrolled wavelength difference between various resonances in the spectrum. This means that resonances from one or more resonators 128 may overlap at nearly the same wavelength (for example, within one linewidth), which in some embodiments may make it difficult to reliably classify which resonances come from which resonator 128 and to track small wavelength shifts. In some embodiments, resonator 128 FSRs may be designed to guarantee that at least one resonance from each resonator 128 is sufficiently separated from all other resonances, enabling accurate classification and tracking of small wavelength shifts. As a non-limiting example in embodiments wherein two multiplexed resonators 128 share a common waveguide of at least an optical waveguide 104a-n, such resonators 128 may be designed to have two or more resonances within a measurable spectrum for each resonator 128. As a further non-limiting example, in embodiments wherein three or more multiplexed resonators 128 share a common waveguide of at least an optical waveguide 104a-n, each such resonator 128 may be designed to have three or more resonances within a measurable spectrum for each resonator 128. As a further embodiment, where four multiplexed resonators 128 share a common waveguide of at least an optical waveguide 104a-n, each resonator 128 may be designed to have at least four resonances within a measurable spectrum for each resonator 128. In an embodiment, design rules as described above may guarantee that at least one resonance for each resonator 128 is not overlapping a resonance from another resonator 128, and its wavelength can be measured precisely. In some embodiments, any overlapping resonances may still be used to help classify which non-overlapping resonances belong to each resonator 128. For example, where multiplexed resonators 128 have different free spectral ranges, and possess two resonances each within a transmission spectrum of each resonator 128, the two resonators 128 may have at most one resonance each that are overlapped with the a resonance of the other resonator 128; this may leave at least one non-overlapped resonance for each resonator 128 that can be tracked accurately.

Continuing to refer to FIG. 1, output signal may have one or more characteristics of a signal from a light source 116a-n. For instance, and without limitation, output signal may have a temporal frequency continuum, simultaneous frequency continuum, or the like, subject to modifications of signals by at least a signal modulator.

Still referring to FIG. 1, plurality of optical modulators may include more than two optical modulators. In an embodiment, and as a non-limiting example, plurality of optical modulators may include at least four optical modulators. Device 100 may alternatively or additionally include a plurality of optical waveguides; that is at least an optical waveguide 104a-n may include a plurality of optical waveguides. Each waveguide of a plurality of optical waveguides may include and/or be optically coupled to its own plurality of optical signal modulators 124 as described above. Similarly, at least a fluidic channel 136 may include a plurality of fluidics channels, which may intersect with plurality of optical signal modulators 124 in any manner that may occur to persons skilled in the art upon reviewing the entirety of this disclosure. For instance, and without limitation if there is more than one fluid lane on the chip, multiplexed biosensors and/or optical signal modulators 124 into the same fluid lane or into different fluid lanes; fluid lanes and biosensors and/or optical signal modulators 124 may form a matrix, wherein each fluidic channel 136 has a biosensor on each waveguide and/or optical signal modulator 124, or the like.

In some embodiments, and with further reference to FIG. 1, during a biosensing function of device, molecules of interest bind to any biosensor and/or optical signal modulator 124 which has been functionalized with a capture reagent such as a capture antibody. This surface reaction may cause a formation of a depletion layer, a fluid region near a chip surface which is depleted of an analyte molecule of interest. This depletion may then cause a reduced signal on a downstream biosensor which is functionalized for the same analyte molecule. In some embodiments, device and/or optical signal modulators 124 may be configured to minimize depletion effects in order to maximize the amount of binding signal obtained from all biosensors that have been functionalized for a given analyte molecule. In some embodiments, to minimize depletion effects a functionalized surface area of the biosensor may be minimalized, for instance and without limitation by minimizing a functionalized length in a direction of the fluid lane. In some embodiments, only the waveguide material of the biosensor may be functionalized by using a surface chemistry that is selective to the waveguide material.

With continued reference to FIG. 1, device 100 includes a receiver module 140 optically coupled to the output of each optical waveguide. Receiver module 140 is an electrooptical device that detects and analyzes signals output from the waveguide; receiver module 140 may detect presence or absence of analytes as described above. Receiver module 140 includes at least a light sensor 144a-n optically coupled to the output of the optical waveguide and configured to detect the plurality of modified signals. As used in this disclosure, a “sensor” is a device that is configured to detect a phenomenon, for example a phenomenon associated with light interacting with at least a detectable marker, as discussed further in this disclosure. A sensor may include any type of sensors, including, and without limitation, sensors configured to detect electrical phenomenon, chemical phenomenon, and/or optical phenomenon. General sensing techniques may include, but are not limited to, using a doped optical waveguide or electrodes near a waveguide to sense an optical change or resistance change, respectively, before or after binding. In some examples, optical changes may be detected using surface plasmon resonances, Mach-Zehnder interferometers 132, spiral waveguides, Bragg gratings, and/or photonic crystals or magnetic dielectric mirrors. In some embodiments, sensors may be collocated with surfaces of waveguides and/or of a substrate that waveguides are integrated into and/or mounted thereto; said another way, surfaces of one or more waveguides may include the sensor or a detection path between the sensor and the at least detectable marker, antigen, or antibody. In one or more embodiments, sensor, such as at least a light sensor 144a-n may be optically connected to and/or in communication with waveguides and/or one or more elements of sample. As used in this disclosure, “communication” is used to refer to a causal or sensed relationship between two relata. Communication may include communication of information and/or data. Communication may also include fluidic communication, where a fluid may be moved between two or more components of device 100. In a nonlimiting example, a photosensor may be said to be in communication with a detectable marker that is reflecting, fluorescing, or otherwise transmitting a light, which the photosensor is detecting, as discussed further below in this disclosure. At least a light sensor 144a-n may be positioned proximate to one or more waveguides. In various embodiments, at least a light sensor 144a-n may include any photon detector. In some cases, an output of optical waveguides, such as an output of a linear waveguide, may be coupled to a photodetector. At least a light sensor 144a-n may include any optical or other sensor, including without limitation a photosensor, a photodetector, a thermopile, a pyrolytic sensor, a photodiode, avalanche photodiode (APD), single photon avalanche photodiode (SPAD), and the like.

Still referring to FIG. 1, receiver module 140 includes circuitry 148 configured to record the plurality of detected modified signals. Receiver module 140 may include circuitry 148 such as without limitation a processor communicatively connected to a memory; for instance, circuitry 148 may include and/or be included in a computing device. As used in this disclosure, “communicatively connected” means connected by way of a connection, attachment, or linkage between two or more relata such as without limitation electronic components, modules, and/or devices which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals there between may include, without limitation, electrical, electromagnetic, magnetic, video, audio, radio and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Continuing to refer to FIG. 1, circuitry 148 may alternatively or additionally be implemented by configuring a hardware device such as a combinatorial or sequential logic circuit, an application-specific integrated circuit (ASIC), a field-programmable gate array (FPGA), or other hardware unit; memory may be attached thereto to further configure the hardware unit using read-only memory (ROM) or any other static or writable memory as described in this disclosure. Alternatively or additionally, hardware units and/or modules may be combined with and/or in communication with a processor, such as without limitation in a system-on-chip architecture wherein some functions are configured by modification or design of hardware circuitry, such as without limitation FPGA circuitry, while others are configured in the form of instructions in memory for one or more processors. As a non-limiting example, any step or combination of steps described herein may be performed entirely using hardware circuit configured to perform such steps either with static memory or rewritable memory. Such steps or combinations of steps may include signing with a digital signature, cryptographically hashing, evaluation of zero-knowledge proofs, or any other specific process described in this disclosure.

With continued reference to FIG. 1, circuitry 148 may be designed and/or configured to perform any method, method step, or sequence of method steps in any embodiment described in this disclosure, in any order and with any degree of repetition. For instance, circuitry 148 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Circuitry 148 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

At least a light sensor 144a-n may be communicatively connected to a circuitry 148 and/or a remote device, such as a user device. Circuitry 148 may include an analog circuit, a computing device, a processor, a microprocessor, and the like. Circuitry 148 may take as input a signal from at least a light sensor 144a-n and process the signal. In some cases, one or more optical elements and/or optical devices may be used to couple EM radiation or light into and/or out of waveguide. For example, and without limitation, coupling lenses having a numerical aperture selected based upon acceptable entrance angle and/or cross-sectional area of waveguide may be used to couple substantially collimated light into waveguide and/or substantially collimate light after exiting waveguide. Circuit may include analog and/or digital circuit elements. Exemplary nonlimiting analog elements include operational amplifiers, comparators, amplification circuits, and the like. In some cases, circuit may include an analog circuit interfaced with a digital circuit, for example and without limitation, by way of an analog to digital (A/D) converter. Alternatively or additionally, an analog circuit may be interfaced with a digital circuit by way of a resistive divider, such as without limitation a Wheatstone bridge. Alternatively or additionally, analog circuit may be interfaced with a digital circuit by way of at least a control terminal of transistors (or other digital elements), which are configured to trip (or otherwise digitally indicate) a certain voltage threshold to configured to be indicative of a change in digital state.

Still referring to FIG. 1, a signal captured from at least a light sensor 144a-n by circuitry 148 may be read and/or recorded in one or more registers such as flip-flop registers and/or may be stored as variables such numerical variables, arrays, vectors, lists, or other data structures storing one or more numerical variables, or the like for further processing. Numerical variables and/or collections thereof may be stored as temporally ordered samples. In some embodiments, where circuitry 148 is clocked, circuitry 148 may record time of reception and combine with detected variables. Such recorded time may be matched with and/or effectively identical to transmission times from light source 116a-n; for instance, circuit may have a second sensor 152 that samples transmitted light of the light source 116a-n essentially simultaneously with capture by at least a light sensor 144a-n of a corresponding sample of output, which given the speed of light and relatively slow time scale of frequency shifts, in the case of a temporal frequency continuum, may be sampling an output that represents a modification of light having the same characteristics of the sensed, emitted light. In an embodiment, light source 116a-n may be programmed to sweep through a range of wavelengths, such as from a minimal wavelength in a tunable range of the light source 116a-n to a maximal wavelength thereof or vice-versa; sweep may be simultaneously recorded and/or matched with output from waveguide by circuitry 148 as described in further detail below.

With continued reference to FIG. 1, receiver module 140 is configured to detect each modified signal of the plurality of modified signals based on its distinct optical characteristic. For instance, and without limitation, receiver module 140 may detect, using optical elements, circuitry 148, and/or configuration thereof, one or more distinct optical characteristics, and use such detection to distinguish an output of one optical signal modulator 124 from that of another optical signal modulator 124. In an embodiment, receiver elements and/or circuitry 148 may demultiplex output to separate it into signals from distinct modulated signals from each optical signal modulator 124 of plurality of optical signal modulators 124. Distinction and/or demultiplexing may be performed by identifying signals produced by different types of optical signal modulators 124 as described above, for instance and without limitation by demultiplexing a modulated signal produced by a resonator 128 from a signal produced by an interferometer 132, as described above. As another non-limiting example, where each optical signal modulator 124 generates a distinct frequency transmission spectrum, receiver module 140 may be configured to detect each modified signal using the distinct frequency transmission spectrum by analyzing a frequency spectrum of an output from the output end of the waveguide. As a non-limiting example, where output has a temporal frequency continuum as described above, receiver module 140 may be configured to analyze a frequency spectrum as a function of the temporal frequency continuum. In some embodiments, receiver module 140 may analyze the frequency spectrum based on stored temporal data of the temporal frequency continuum. For instance and without limitation, a tunable laser 120a-n or other light source 116a-n having a temporal frequency continuum output may be calibrated and circuitry 148 may generate and/or receive modulation signal to laser, and/or may have access to stored instruction(s) according to which the laser will be modulated. As a result, circuitry 148 may be configured to construct a map of input signals to frequencies, enabling it to describe the frequency at which each detected sample was received.

Still referring to FIG. 1, receiver module 140 may include an optical frequency discriminator 156. An “optical frequency discriminator,” as used herein, is an optical component that is used to determine a frequency and/or frequency spectrum of an optical signal. Optical frequency discriminator 156 may be preceded by a splitter that divides output into a first signal for optical frequency discriminator 156 and a second signal for at least a light sensor 144a-n. Optical frequency discriminator 156 may include a discriminator optical component 160 that generates a frequency-dependent pattern based on frequency and/or frequency spectrum of output, which pattern may be detected by at least a discriminator sensor 164 or a plurality thereof. Discriminator optical component 160 may include, as a non-limiting example, an interferometer 132 that produces an interference pattern, fringe or the like based on an instantaneous frequency and/or frequency spectrum of output, coupled with sensors that detect and record such interference pattern, fringe, or the like. As a further non-limiting example, optical frequency discriminator 156 may include a grating and/or prismatic device that maps frequencies to spatial locations, and a sensor array that detects light at each such location. Optical frequency discriminator 156 may include one or more optical filters such as dichroic mirrors, resonator 128 filters, or the like, which may be arranged in a tree of splitters or the like leading to individual sensors of at least a discriminator sensor 164 to determine an intensity at each wavelength and/or frequency.

Still referring to FIG. 1, where each modified signal of the plurality of modified signals includes at least one frequency domain extremum, such as without limitation a frequency domain extremum that does not overlap with an extremum of any other modified signal of the plurality of modified signals, receiver module 140 may be configured to detect the frequency domain extremum. In some embodiments, extrema may be detected by comparing intensities detected per frequency to threshold intensities, to threshold increases or decreases in intensity relative to intensities detected at other frequencies, or the like; for instance, an increase or decrease of more than a threshold number of decibels when moving from one frequency to another, such as is used in a frequency profile of a filter such as a Butterworth or elliptical high-pass, low-pass, or bandpass filter, a decibel level above or below a preconfigured threshold, or the like may be interpreted by circuitry 148 as representing extrema; a frequency at which such extrema were detected may be recorded by circuitry 148 for further analysis and/or analyte detection. Alternatively or additionally, a frequency domain pattern and/or waveform of output and/or one or more components thereof may be recorded, such a combination of multiple sinusoidal waveforms representing a plurality of interferometer 132 outputs, which may be further processed as described below.

With continued reference to FIG. 1, circuitry 148 of receiver module 140 may perform one or more analytical tasks to identify and/or isolate modulated signals from particular optical signal modulators 124. For instance and without limitation, circuitry 148 of receiver may separate a frequency-domain representation of output into individual sinusoidal components using Fourier transform, for instance and without limitation by employing a hardware and/or software-implemented fast Fourier transform (FFT); this may be used, without limitation, to separate interference patterns from different interferometers 132 from each other, which can then be analyzed further to determine presence or absence of an analyte. As a further non-limiting example, Fourier series may be used to separate and/or distinguish signal components of frequency domain, for instance by transforming the Fourier series, separating peaks representing periods of sinusoidal patterns, reverse Fourier transforming the separated peaks, and analyzing and/or processing the resulting separated sinusoidal patterns separately. Signal processing techniques may be used to automatically classify which resonances belong to which resonator 128, and to track wavelength shifts due to molecules binding to the biosensors. For instance, and without limitation, receiver module 140 may be configured to analyze a frequency spectrum of output by mapping extrema to optical modulators. This may be done using lookup tables, control flow logic, and/or stored values or value ranges for comparisons.

Alternatively or additionally, pairwise comparisons of pairs of resonance wavelengths from the spectrum can be used to build up a probabilistic determination of which resonances map to which resonator 128 based on FSR. Other weighting criteria may also be used, such as the extinction ratio or linewidth of the resonances. For instance, and without limitation, ratio or degree of difference between an extremum and a surrounding signal in the frequency domain may be associated with a particular modulator, such as without limitation a particular resonator and/or interferometer, in training data used to train a machine-learning model, neural network, clustering model, or the like, in calibration data used to calibrate receiver module 140, and/or as a criterion to identify a signal element produced by the particular resonator and/or interferometer. As a further example, training data, calibration data, and/or other data may be used to associate a particular modulator with a particular characteristic of a modulated signal, such as a width of an extremum such as a “spike” or “notch” created by a resonator, or the like.

Alternatively or additionally, and still referring to FIG. 1, one or more signal processing techniques and/or machine-learning techniques may be used to partition signal features such as resonance wavelengths or other distinct optical characteristics into partitions representing each optical signal modulator 124 of a plurality of optical signal modulators 124 on a common waveguide. In a non-limiting example, a clustering algorithm such as k-means clustering may be used to partition n observed resonance wavelengths into k clusters that represent the multiplexed resonators 128. A “k-means clustering algorithm” as used in this disclosure, includes cluster analysis that partitions n observations or unclassified cluster data entries into k clusters in which each observation or unclassified cluster data entry belongs to the cluster with the nearest mean. “Cluster analysis” as used in this disclosure, includes grouping a set of observations or data entries in way that observations or data entries in the same group or cluster are more similar to each other than to those in other groups or clusters. Cluster analysis may be performed by various cluster models that include connectivity models such as hierarchical clustering, centroid models such as k-means, distribution models such as multivariate normal distribution, density models such as density-based spatial clustering of applications with nose (DBSCAN) and ordering points to identify the clustering structure (OPTICS), subspace models such as biclustering, group models, graph-based models such as a clique, signed graph models, neural models, and the like. Cluster analysis may include hard clustering whereby each observation or unclassified cluster data entry belongs to a cluster or not. Cluster analysis may include soft clustering or fuzzy clustering whereby each observation or unclassified cluster data entry belongs to each cluster to a certain degree such as for example a likelihood of belonging to a cluster; for instance, and without limitation, a fuzzy clustering algorithm may be used to identify clustering of elements of a first type or category with elements of a second type or category, and vice versa. Cluster analysis may include strict partitioning clustering whereby each observation or unclassified cluster data entry belongs to exactly one cluster. Cluster analysis may include strict partitioning clustering with outliers whereby observations or unclassified cluster data entries may belong to no cluster and may be considered outliers. Cluster analysis may include overlapping clustering whereby observations or unclassified cluster data entries may belong to more than one cluster. Cluster analysis may include hierarchical clustering whereby observations or unclassified cluster data entries that belong to a child cluster also belong to a parent cluster.

With continued reference to FIG. 1, circuitry 148 may generate a k-means clustering algorithm receiving unclassified data and output a definite number of classified data entry clusters wherein the data entry clusters each contain cluster data entries. K-means algorithm may select a specific number of groups or clusters to output, identified by a variable “k.” Generating a k-means clustering algorithm includes assigning inputs containing unclassified data to a “k-group” or “k-cluster” based on feature similarity. Centroids of k-groups or k-clusters may be utilized to generate classified data entry cluster. K-means clustering algorithm may select and/or be provided “k” variable by calculating k-means clustering algorithm for a range of k values and comparing results. K-means clustering algorithm may compare results across different values of k as the mean distance between cluster data entries and cluster centroid. K-means clustering algorithm may calculate mean distance to a centroid as a function of k value, and the location of where the rate of decrease starts to sharply shift, this may be utilized to select a k value. Centroids of k-groups or k-cluster include a collection of feature values which are utilized to classify data entry clusters containing cluster data entries. K-means clustering algorithm may act to identify clusters of closely related data, which may be provided with user cohort labels; this may, for instance, generate an initial set of user cohort labels from an initial set of data, and may also, upon subsequent iterations, identify new clusters to be provided new labels, to which additional data may be classified, or to which previously used data may be reclassified.

With continued reference to FIG. 1, generating a k-means clustering algorithm may include generating initial estimates for k centroids which may be randomly generated or randomly selected from unclassified data input. K centroids may be utilized to define one or more clusters. K-means clustering algorithm may assign unclassified data to one or more k-centroids based on the squared Euclidean distance by first performing a data assigned step of unclassified data. K-means clustering algorithm may assign unclassified data to its nearest centroid based on the collection of centroids ci of centroids in set C. Unclassified data may be assigned to a cluster based on [symbol] dist(ci,x)², where argmin includes argument of the minimum, ci includes a collection of centroids in a set C, and dist includes standard Euclidean distance. K-means clustering module may then recompute centroids by taking mean of all cluster data entries assigned to a centroid's cluster. This may be calculated based on ci=1/|Si|Σxi [symbol] Si^{xi}. K-means clustering algorithm may continue to repeat these calculations until a stopping criterion has been satisfied such as when cluster data entries do not change clusters, the sum of the distances have been minimized, and/or some maximum number of iterations has been reached.

Still referring to FIG. 1, k-means clustering algorithm may be configured to calculate a degree of similarity index value. A “degree of similarity index value” as used in this disclosure, includes a distance measurement indicating a measurement between each data entry cluster generated by k-means clustering algorithm and a selected element. Degree of similarity index value may indicate how close a particular combination of elements is to being classified by k-means algorithm to a particular cluster. K-means clustering algorithm may evaluate the distances of the combination of elements to the k-number of clusters output by k-means clustering algorithm. Short distances between an element of data and a cluster may indicate a higher degree of similarity between the element of data and a particular cluster. Longer distances between an element and a cluster may indicate a lower degree of similarity between a elements to be compared and/or clustered and a particular cluster.

With continued reference to FIG. 1, k-means clustering algorithm may select a classified data entry cluster as a function of the degree of similarity index value. In an embodiment, k-means clustering algorithm may select a classified data entry cluster with the smallest degree of similarity index value indicating a high degree of similarity between an element and the data entry cluster. Alternatively or additionally k-means clustering algorithm may select a plurality of clusters having low degree of similarity index values to elements to be compared and/or clustered thereto, indicative of greater degrees of similarity. Degree of similarity index values may be compared to a threshold number indicating a minimal degree of relatedness suitable for inclusion of a set of element data in a cluster, where degree of similarity indices a-n falling under the threshold number may be included as indicative of high degrees of relatedness. The above-described illustration of feature learning using k-means clustering is included for illustrative purposes only, and should not be construed as limiting potential implementation of feature learning algorithms; persons skilled in the art, upon reviewing the entirety of this disclosure, will be aware of various additional or alternative feature learning approaches that may be used consistently with this disclosure.

Alternatively or additionally, clustering may be performed by evolutionary algorithms such as genetic algorithms and/or particle swarm optimization, where a plurality of candidate solutions corresponding to potential partitions of signal features such as resonance wavelengths or other distinct optical characteristics may be iteratively modified and compared to convergence tests until the plurality of candidate solutions converges on an optimal partition of signal features. Clustering and/or partitioning may alternatively or additionally be performed using an encoder, where an “encoder” is a neural network that maps input elements, such as without limitation features of a sensed output of the waveguide, to embeddings or vectors representing distinct signal features such as resonance wavelengths or other distinct optical characteristics to vectors that can be associated using one or more tests of vector similarity such as without limitation geometric similarity including cosine similarity. In some embodiments, tests of vector similarity may be used to associate vectors with one another, and hence associate signal elements output by the same optical signal modulator 124 to one another while disassociating, for instance through identifying dissimilar vectors, outputs from distinct optical signal modulators 124.

With continued reference to FIG. 1, a “vector” as defined in this disclosure is a data structure that represents an element of a vector space; a vector may alternatively or additionally be represented as an element of a vector space, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. A vector may be represented as an n-tuple of values, where n is one or more values, as described in further detail below; a vector may alternatively or additionally be represented as an element of a vector space, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent, for instance as measured using cosine similarity as computed using a dot product of two vectors; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {a}_{i}^{2}} ,$$

where ai is attribute number i of the vector. Scaling and/or normalization may function to make vector comparison independent of absolute quantities of attributes, while preserving any dependency on similarity of attributes. A two-dimensional subspace of a vector space may be defined by any two orthogonal vectors contained within the vector space. Two-dimensional subspace of a vector space may be defined by any two orthogonal and/or linearly independent vectors contained within the vector space; similarly, an n-dimensional space may be defined by n vectors that are linearly independent and/or orthogonal contained within a vector space. A vector's “norm’ is a scalar value, denoted ∥a∥ indicating the vector's length or size, and may be defined, as a non-limiting example, according to a Euclidean norm for an n-dimensional vector a as:

$$‖ a ‖ = \sqrt{{\sum }_{i = 0}^{n} {a}_{i}^{2}}$$

Without limitation, and still referring to FIG. 1, one or more clustering, encoding, and/or partitioning algorithms as described above may be implemented using machine-learning techniques and/or neural networks as described in further detail below. Algorithms, machine-learning models, and/or neural networks may be trained and/or pretrained to identify centroids in vector spaces and/or other clustering and/or partitioning protocols and/or designs. For instance, such algorithms, machine-learning models, and/or neural networks may be pretrained on a set of optical signal modulators 124 to sort signal outputs therefrom to centroids representing untested and/or tested resonances; this may involve both supervised processes wherein such algorithms, machine-learning models, and/or neural networks may be pretrained to recognize and/or sort optical characteristics such as distinct optical characteristics and/or to sort such optical characteristics to centroids, clusters, partition elements, vectors, or the like. Algorithms, machine-learning models, and/or neural networks may also be subjected to one or more rounds of supervised training using, e.g., input signals correlated to correct association to centroids, clusters, partition elements, and/or vectors, where incorrection identification and/or a measure (e.g. mean squared error) of distance per a corresponding distance metric to the correct centroid, partition element, and/or vector as error for tuning of parameters such as coefficients, biases, or the like and/or for back-propagation. Training may be performed, without limitation, across different lots or designed types and/or characteristics of optical signal modulators 124 during manufacturing; a retraining or tuning to converge to specific optical signal modulators 124 as deployed in a given sensor array and/or device may be performed by circuitry 148 by, e.g., ensuring that resonators 128 and/or other optical signal modulators 124 without analytes are matched to centroids, clusters, partitions, and/or vectors within a preconfigured degree of convergence. Pretrained and/or trained algorithms, machine-learning models, and/or neural networks may be received by and/or deployed on circuitry 148 using any process or process steps described below. In some embodiments, any overlapping resonances may still be used to help classify which non-overlapping resonances belong to each resonator 128. For instance, and without limitation, such overlapping resonances may be used as components of inputs to classifiers, in combination with non-overlapping resonances.

Further referring to FIG. 1, device may be designed to perform one or more calibration actions and/or processes prior to use for detection of analytes. For instance, and without limitation, device 100 may be designed or configured to perform pretraining and/or to test its own centroid identification as described above. Device 100 may alternatively or additionally be designed to have one or more optical channels which only contain one biosensor. This single biosensor may provide an unambiguous reference optical characteristic, such as a reference FSR, which can be used as a calibration parameter for the expected optical characteristics and/or FSRs of other optical signal modulators 124 and/or resonators 128 incorporated in device. Calibration parameter may in turn be used to retrain algorithms, machine-learning models, and/or neural networks as described above.

Still referring to FIG. 1, receiver module 140 is configured to determine presence of each of the at least a biological analyte based on a respective detected modified signal. In an embodiment, detection may be performed by identifying a shift in frequency of one or more extrema such as resonances, where a shift in frequency may be compared to a preconfigured threshold. Preconfigured threshold may be tuned, in a non-limiting example, using statistical processes such as regression analysis and/or machine learning to identify an optimally reliable threshold; threshold selection may be optimized to eliminate false positives, eliminate false negatives, and/or to reduce each below a selected likelihood and/or relative frequency in testing. Analyte detection may be performed, without limitation, according to any process or process steps described in this disclosure and/or in disclosures incorporated by reference herein.

Continuing to refer to FIG. 1, one or more algorithms, machine-learning models, and/or neural networks may be trained to identify analytes, for instance and without limitation, pairs of signals from optical signal modulators 124 that have not been exposed to analytes and signals from optical signal modulators 124 that have been exposed to analytes may be used to train a machine-learning model to detect signals that are consistent with the presence of analytes. Training data may be sorted and/or classified to be specific to categories or lots of optical signal modulators 124, to types of chemical substrates, markers, nanoparticles, or the like, and/or to types of analytes, to produce machine-learning models and/or neural networks specifically trained to detect analytes in situations matching such categories of training data.

In some embodiments, receiver module 140 and/or at least a light source 116a-n may be incorporated in a first component such as a reader device, while plurality of optical signal modulators 124 and/or plurality of fluidic channels 136 may be incorporated in a second component such as a sensor device; second component may be disposable, while other embodiments thereof may be connected to reader component after each use and disposal of a previous sensor component. Aspects of reader component may be implemented, without limitation, as described in U.S. Nonprovisional application Ser. No. 19/079,377, filed on Mar. 21, 2023 with attorney docket number 1214-009USU1 and entitled “OPTICAL READER DEVICE FOR MULTIPLEXED DIAGNOSTIC SYSTEMS AND METHODS OF USE,” the entirety of which is incorporated herein by reference. Aspects of sensor component may be implemented, without limitation, as described in U.S. Nonprovisional application Ser. No. 18/126,014, filed on Mar. 24, 2023 with attorney docket number 1214-010USU1 and entitled “PHOTONIC BIOSENSOR FOR MULTIPLEXED DIAGNOSTICS AND A METHOD OF USE,” the entirety of which is incorporated herein by reference and in U.S. Provisional Application Ser. No. 63/742,696, filed on Jan. 7, 2025 with attorney docket number 1214-046USP1 and entitled “MICROFLUIDICS ARCHITECTURE FOR A DISPOSABLE CARTRIDGE IN A HOME TESTING DEVICE,” the entirety of which is incorporated herein by reference. At least an optical waveguide 104a-n may optically connect to reader component, light source 116a-n, and/or receiver module 140 by way of one or more couplings to waveguides connected to light source 116a-n and/or receiver module 140. Coupling may be accomplished, without limitation, as disclosed in U.S. Nonprovisional application Ser. No. 19/079,377, filed on Mar. 13, 2025 with attorney docket number 1214-104USU1 and entitled “APPARATUS AND METHOD OF MANUFACTURE FOR A PLUGGABLE INTER-CHIPE OPTICAL CONNECTOR,” the entirety of which is incorporated herein by reference, and in U.S. Nonprovisional application Ser. No. 18/000,818, filed on Dec. 12, 2022, having attorney docket number 1214-102USU1, and entitled “SYSTEMS AND METHODS FOR PHOTONIC CHIP COUPLING,” the entirety of which is incorporated herein by reference. One or more fluidic channels 136 located in a sensor component may be connected fluidically to one or more fluidics channels of a reader component or other portion of a system and/or device incorporating components as described in this disclosure; fluids may be moved through fluidics channels and/or channels connected thereto by active or passive means, for instance and without limitation as described in U.S. Nonprovisional application Ser. No. 17/859,932, filed on Jul. 7, 2022 with attorney docket number 1214-005USU1 and entitled “SYSTEMS AND METHODS FOR FLUID SENSING USING PASSIVE FLOW,” the entirety of which is incorporated herein by reference, and in U.S. Nonprovisional application Ser. No. 18/107,135, filed on Feb. 18, 2023 with attorney docket number 1214-007USU1 and entitled “APPARATUS AND METHODS FOR ACTUATING FLUIDS IN A BIOSENSOR CARTRIDGE,” the entirety of which is incorporated herein by reference.

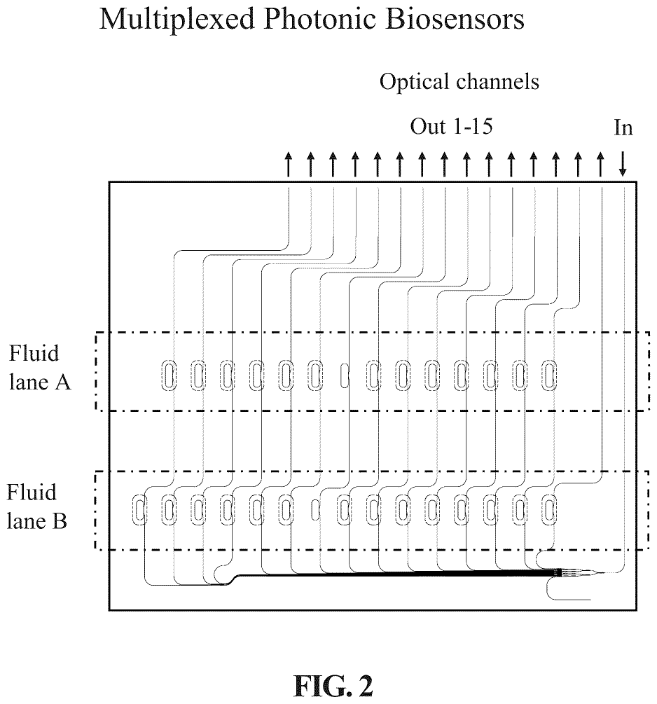

Referring now to FIG. 2, an exemplary embodiment of a portion of device 100 is illustrated. Device may include up to two resonators 128 200 multiplexed onto each common optical bus 204 of at least an optical waveguide 104a-n, which leads to an optical output channel. Output channels may be coupled to optical fibers; coupling may be performed in any manner described in this disclosure. Output channels may lead to photodetectors. Alternatively or additionally, photodetectors may be integrated on the same chip as resonators 128 200. In an exemplary embodiment, biosensors in fluid lane A may be designed to have free spectral range A, and biosensors in fluid lane B may be designed to have free spectral range B; alternatively or additionally, free spectral ranges and/or optical characteristics in a given fluid lane may differ from one another. Optical characteristics and/or type of optical signal modulator 124 may be selected based on ability to detect a given analyte more effectively; for instance, and without limitation, one analyte may have a more pronounced effect on an interference pattern with an interferometer 132 than on a resonant frequency of a resonator 128, resulting in selection of an interferometer 132 to test for that analyte, whereas another analyte may have a more pronounced effect on a resonance than on interference patterns, leading to selection of a resonator 128.

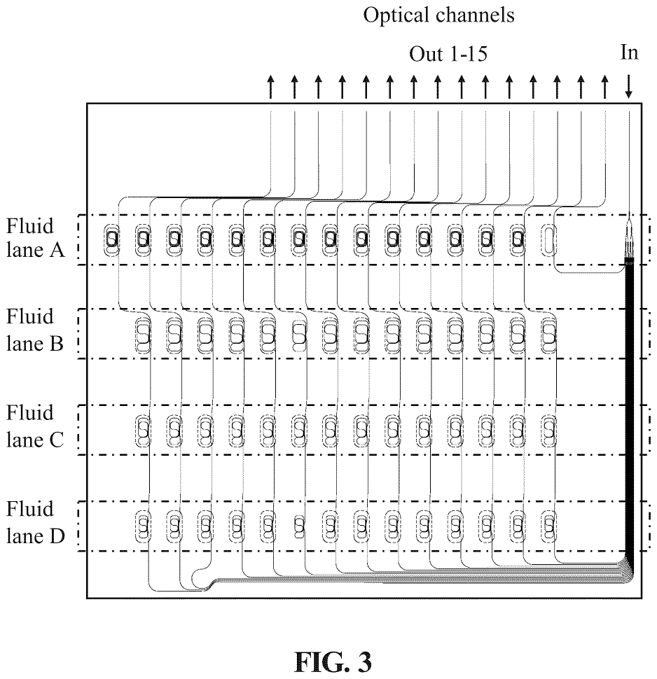

Referring now to FIG. 3. An exemplary embodiment of a portion of device 100 having up to four resonators 128 or other optical signal modulators 124 per optical bus. Each optical bus may have and/or be couple to an outlet; there may be one light sensor per optical bus.

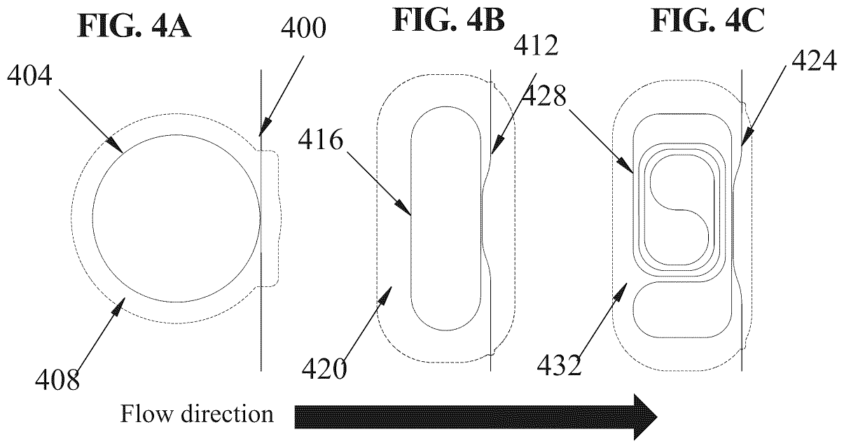

Referring now to FIG. 4A, an exemplary embodiment of an optical signal modulator (here illustrated for exemplary purposes as a ring resonator 400 in close proximity to a waveguide 404) is depicted having a larger trench 408 around the optical signal modulator. Trench 408 may be depressed and/or recessed relative to a surface of a surrounding chip. Some or all of surface area in trench 408 may be functionalized to bind and/or react with analyte. Trench 408 may have any horizontal cross-sectional form, including a substantially circular form, a substantially square form, and/or any combination of polygonal and curved elements.

Referring now to FIG. 4B, in other embodiments, a racetrack resonator 416 coupled to an optical bus 416 may be designed to reduce a functionalized length along a fluid lane compared to a microring resonator while still maintaining the desired free spectral range; a corresponding trench 420 may be approximately shaped like the racetrack resonator with fluid flow traveling across the trench's shorter dimension. This may reduce attenuation effects within fluid while maintaining a strong effect on resonance and/or other optical characteristics.

Referring now to FIG. 4C, in other embodiments, a spiral resonator 428 coupled to an optical bus 424 can be designed to provide a long path length resonator to achieve a small free spectral range target within a minimal functionalized length along the fluid lane; as before, a corresponding trench 432 may be approximately shaped like an outer perimeter of the spiral resonator 428 with fluid flow traveling across the trench's shorter dimension. This may reduce attenuation effects within fluid while maintaining a strong effect on resonance and/or other optical characteristics.

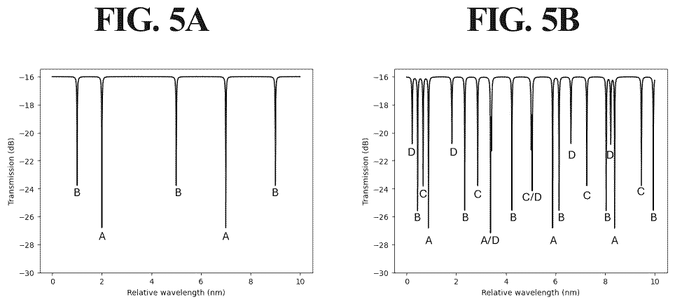

FIG. 5A shows simulated transmission spectra in a non-limiting exemplary embodiment for multiplexed resonators 128 on a common optical bus. Two multiplexed resonators 128′ outputs are depicted as designed so that each resonator 128 has at least two resonances within the spectrum: bandwidth=10 nm, FSR_A=5 nm, FSR_B=4 nm. FIG. 5B shows simulated transmission spectra in an exemplary embodiment where four multiplexed resonators 128 are designed so that each resonator 128 has at least 4 resonances within the spectrum: bandwidth=10 nm, FSR_A=2.5 nm, FSR_B=2.2 nm, FSR_C=1.9 nm, FSR_D=1.6 nm. In both FIG. 5A and FIG. 5B, resonances have been labeled with which resonator 128 they correspond to. Labels such as “A/D” indicate where two resonances are overlapping or close together such that they may be difficult to accurately label which resonance belongs to which resonator 128 or to detect small wavelength shifts. In an embodiment, a design similar to that shown may guarantee that at least one resonance from each resonator 128 is sufficiently separated from all other resonances such that this at least one resonance wavelength can be accurately classified and tracked.

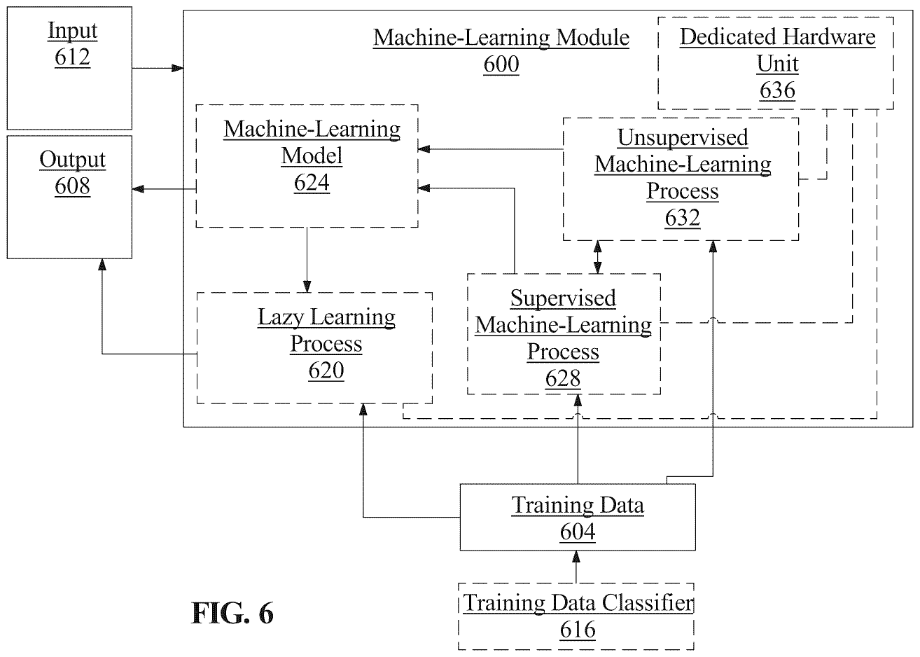

Referring now to FIG. 6, an exemplary embodiment of a machine-learning module 600 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 604 to generate an algorithm instantiated in hardware or software logic, data structures, and/or functions that will be performed by a computing device/module to produce outputs 608 given data provided as inputs 612; this is in contrast to a non-machine learning software program where the commands to be executed are determined in advance by a user and written in a programming language.

Still referring to FIG. 6, “training data,” as used herein, is data containing correlations that a machine-learning process may use to model relationships between two or more categories of data elements. For instance, and without limitation, training data 604 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 604 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 604 according to various correlations; correlations may indicate causative and/or predictive links between categories of data elements, which may be modeled as relationships such as mathematical relationships by machine-learning processes as described in further detail below. Training data 604 may be formatted and/or organized by categories of data elements, for instance by associating data elements with one or more descriptors corresponding to categories of data elements. As a non-limiting example, training data 604 may include data entered in standardized forms by persons or processes, such that entry of a given data element in a given field in a form may be mapped to one or more descriptors of categories. Elements in training data 604 may be linked to descriptors of categories by tags, tokens, or other data elements; for instance, and without limitation, training data 604 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 6, training data 604 may include one or more elements that are not categorized; that is, training data 604 may not be formatted or contain descriptors for some elements of data. Machine-learning algorithms and/or other processes may sort training data 604 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 604 to be made applicable for two or more distinct machine-learning algorithms as described in further detail below. Training data 604 used by machine-learning module 600 may correlate any input data as described in this disclosure to any output data as described in this disclosure.

Further referring to FIG. 6, training data may be filtered, sorted, and/or selected using one or more supervised and/or unsupervised machine-learning processes and/or models as described in further detail below; such models may include without limitation a training data classifier 616. Training data classifier 616 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 600 may generate a classifier using a classification algorithm, defined as a processes whereby a computing device and/or any module and/or component operating thereon derives a classifier from training data 604. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 616 may classify elements of training data to particular categories of optical signal modulator 124, analytes, materials used for detection, or the like.

Still referring to FIG. 6, a computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. A computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 6, a computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 6, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 6, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. A computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 6, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 6, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 6, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 6, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 6, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 6, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 6, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{IQR} .$$

Persons skilled in the art, upon reviewing the entirety of this disclosure, will be aware of various alternative or additional approaches that may be used for feature scaling.

Further referring to FIG. 6, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 6, machine-learning module 600 may be configured to perform a lazy-learning process 620 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 604. Heuristic may include selecting some number of highest-ranking associations and/or training data 604 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 6, machine-learning processes as described in this disclosure may be used to generate machine-learning models 624. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 624 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 624 may be generated by creating an artificial neural network, such as a convolutional neural network comprising an input layer of nodes, one or more intermediate layers, and an output layer of nodes. Connections between nodes may be created via the process of “training” the network, in which elements from a training data 604 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 6, machine-learning algorithms may include at least a supervised machine-learning process 628. At least a supervised machine-learning process 628, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs as described in this disclosure as inputs, outputs as described in this disclosure as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 604. Persons skilled in the art, upon reviewing the entirety of this disclosure, will be aware of various possible variations of at least a supervised machine-learning process 628 that may be used to determine relation between inputs and outputs. Supervised machine-learning processes may include classification algorithms as defined above.

With further reference to FIG. 6, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Continuing to refer to FIG. 6, evaluation of error function and/or other comparison results may include comparison of each of error function and/or other comparison results to a maximum single error threshold; in other words, a criterion of evaluation may include performing iterative retraining if any single comparison and/or error function output exceeds maximum single error threshold or if a count of single comparison and/or error function outputs exceeding single error threshold exceeds a threshold number and/or proportion of overall error function and/or other comparison results. Alternatively or additionally, evaluation of error function and/or other comparison results may include comparison of an aggregated plurality of error function and/or other comparison results to an aggregate error threshold; in other words, a criterion of evaluation may include performing iterative retraining if a result of averaging or otherwise aggregating a plurality such as some or all evaluated function and/or other comparison results exceeds aggregate error threshold. Aggregation may be performed in any manner of aggregation described in this disclosure and/or any combination thereof. Criteria for evaluations may be evaluated separately such that failing any one criterion causes iterative retraining; alternatively or additionally evaluation results may be combined according to one or more logical or other rules.

As a non-limiting, illustrative example, and still referring to FIG. 6, where outputs to be compared by error function are numerical values, error function may include subtraction of one from the other to derive an absolute value and/or mean squared error. Where outputs and/or training examples are represented as a binary classification, an error function may include a hinge loss function, sigmoid cross entropy loss function, weighted cross entropy loss function, or the like. Where output and/or exemplary output in a training set is a classification to three or more values, error function may include a softmax cross entropy loss function, a sparse cross entropy loss function, a Kullback-Leibler divergence loss function, or the like. Where both retaining and training with include supervised training, retraining may use a different error function, different weight update functions and/or parameters, or the like than in the training stage. For instance, and without limitation, when a previous iterative retraining process included training using examples from until a first convergence threshold and/or epsilon value and/or neighborhood is met, a subsequent iterative retraining process may include a lower convergence threshold, a smaller value of epsilon, or the like. Iterative retraining may include using one or more examples that were not used in any previous training and/or retraining process; for instance, where convergence was initially and/or previously achieved using a first subset of examples a subsequent retraining process may use examples from a second subset of examples, which may be wholly disjoint from first subset and/or have one or more elements that are not found in first subset.

Still referring to FIG. 6, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 6, machine learning processes may include at least an unsupervised machine-learning processes 632. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 632 may not require a response variable; unsupervised processes 632 may be used to find interesting patterns and/or inferences between variables, to determine a degree of correlation between two or more variables, or the like.

Still referring to FIG. 6, machine-learning module 600 may be designed and configured to create a machine-learning model 624 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 6, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 6, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 6, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 6, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 6, one or more processes or algorithms described above may be performed by at least a dedicated hardware unit 636. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 636 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 636 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 636 to perform one or more operations described herein, such as evaluation of model and/or algorithm outputs, one-time or iterative updates to parameters, coefficients, weights, and/or biases, and/or any other operations such as vector and/or matrix operations as described in this disclosure.

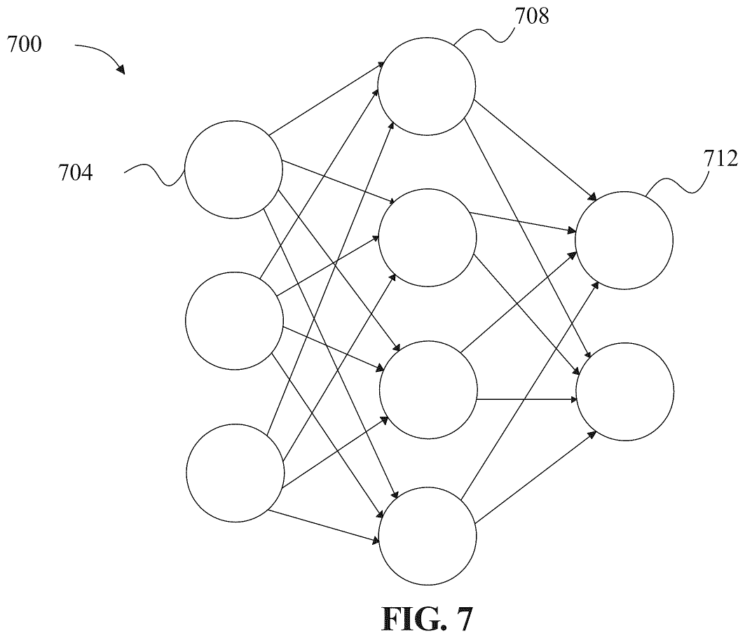

Referring now to FIG. 7, an exemplary embodiment of neural network 700 is illustrated. A neural network 700 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 704, one or more intermediate layers 708, and an output layer of nodes 712. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

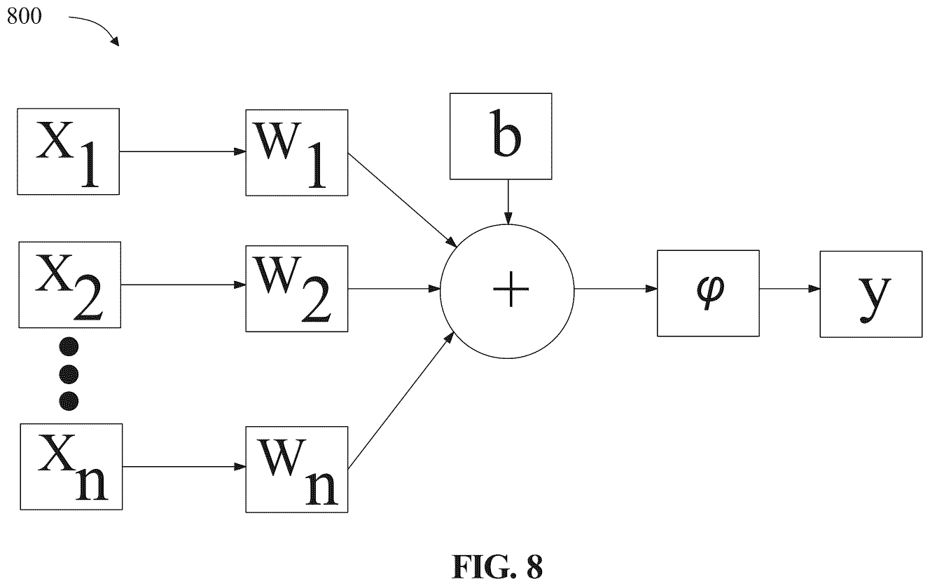

Referring now to FIG. 8, an exemplary embodiment of a node 800 of a neural network is illustrated. A node may include, without limitation, a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as ƒ(x)=tanh²(x), a rectified linear unit function such as ƒ(x)=max(0,x), a “leaky” and/or “parametric” rectified linear unit function such as ƒ(x)=max(ax,x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as ƒ(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ, or of other coefficients and/or parameters of an activation function, may be determined by training a neural network using training data, which may be performed using any suitable process as described above. Each weight in a neural network may, without limitation, be updated and/or tuned, based on an error function J, using a backpropagation updating method, such as:

$${w}_{new} = {w}_{old} - α \frac{dJ}{dw}$$

where w_{new} is the updated weight value, w_{old} is the previous weight value, α is a parameter to set the learning rate, and

$$\frac{dJ}{dw}$$

is the partial derivative of with respect to weight w.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

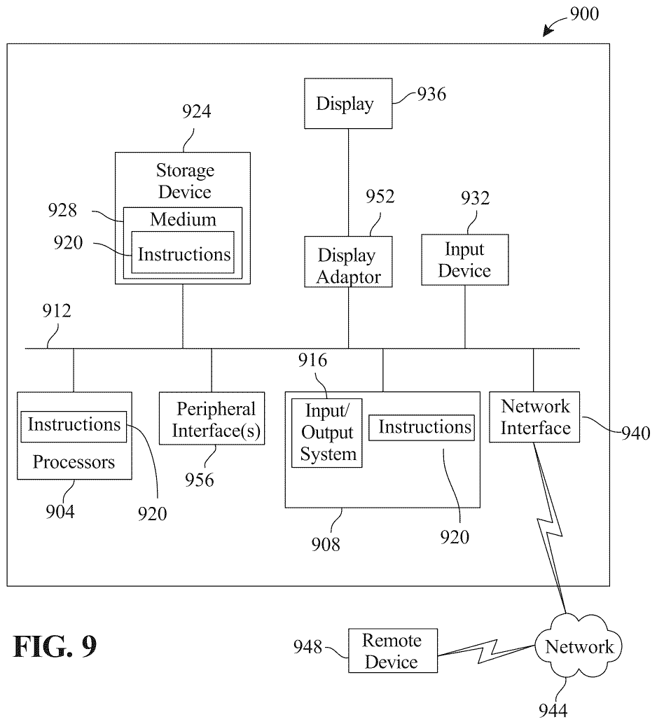

FIG. 9 shows a diagrammatic representation of one embodiment of a computing device in the exemplary form of a computer system 900 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 900 includes a processor 904 and a memory 908 that communicate with each other, and with other components, via a bus 912. Bus 912 may include any of several types of bus structures including, but not limited to, a memory bus, a memory controller, a peripheral bus, a local bus, and any combinations thereof, using any of a variety of bus architectures.

Processor 904 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 904 may be organized according to Von Neumann and/or Harvard architecture as a non-limiting example. Processor 904 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC). Each processor and/or processor core may perform a state transition, instruction, and/or instruction step during a period of a “clock,” or a regular oscillator that generates periodic output waveform, such as a square wave, having a regular period; different processors and/or cores may have distinct clocks. A processor may operate as and/or include a processing unit that performs instruction inputs, arithmetic operations, logical operations, memory retrieval operations, memory allocation operations, and/or input and output operations; a circuitry or module within a processor may determine which of the above-described functions a processor and/or unit within a processor will perform on a given clock cycle. A processor may include a plurality of processing units or “cores,” each of which performs the above-described actions; multiple cores may work on disparate instruction sets and/or may work in parallel. A single core may also include multiple arithmetic, logic, or other units that can work in parallel with each other. Parallel computing between and/or within processors and/or cores may include multithreading processes and/or protocols such as without limitation Tomasulo's algorithm. As used in this disclosure, “a processor,” and/or “configuring a processor,” is equivalent for the purposes of this disclosure to at least a processor, a plurality of processors, and/or a plurality of processor cores, and/or programming at least a processor, a plurality of processors, and/or a plurality of processor cores, which may be configured to operate on instructions in parallel and/or sequentially according to multithreading algorithms, parallel computing, load and/or task balancing, and/or virtualization, for instance and without limitation as described below.

Memory 908 may include various components (e.g., machine-readable media) including, but not limited to, a random-access memory component, a read only component, and any combinations thereof. In one example, a basic input/output system 916 (BIOS), including basic routines that help to transfer information between elements within computer system 900, such as during start-up, may be stored in memory 908. Memory 908 may also include (e.g., stored on one or more machine-readable media) instructions (e.g., software) 920 embodying any one or more of the aspects and/or methodologies of the present disclosure. In another example, memory 908 may further include any number of program modules including, but not limited to, an operating system, one or more application programs, other program modules, program data, and any combinations thereof. Memory 908 may include a primary memory and a secondary memory. “Primary memory,” which may be implemented, without limitation as “random access memory” (RAM), is memory used for temporarily storing data for active use by a processor. In one or more embodiments, during use of the computing device, instructions and/or information may be transmitted to primary memory wherein information may be processed. In one or more embodiments, information may only be populated within primary memory while a particular software is running. In one or more embodiments, information within primary memory is wiped and/or removed after the computing device has been turned off and/or use of a software has been terminated. In one or more embodiments, primary memory may be referred to as “Volatile memory” wherein the volatile memory only holds information while data is being used and/or processed. In one or more embodiments, volatile memory may lose information after a loss of power.

Computer system 900 may also include a storage device 924. Examples of a storage device (e.g., storage device 924) include, but are not limited to, a hard disk drive, a magnetic disk drive, an optical disc drive in combination with an optical medium, a solid-state memory device, and any combinations thereof. Storage device 924 may be connected to bus 912 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 924 (or one or more components thereof) may be removably interfaced with computer system 900 (e.g., via an external port connector (not shown)). Particularly, storage device 924 and an associated machine-readable medium 928 may provide nonvolatile and/or volatile storage of machine-readable instructions, data structures, program modules, and/or other data for computer system 900. In some embodiments, storage device 924 and/or devices “Secondary memory” also known as “storage,” “hard disk drive” and the like for the purposes of this disclosure is a long-term storage device in which an operating system and other information is stored; operating system and/or main program instructions may alternatively or additionally be stored in hard-coded memory ROM, or the like. In one or remote embodiments, information may be retrieved from secondary memory and copied to primary memory during use. In one or more embodiments, secondary memory may be referred to as non-volatile memory wherein information is preserved even during a loss of power. In some embodiments, data from secondary memory is transferred to primary memory before being accessed by a processor. In one or more embodiments, data is transferred from secondary to primary memory wherein circuitry 102 may access the information from primary memory. In one example, software 920 may reside, completely or partially, within machine-readable medium 928. In another example, software 920 may reside, completely or partially, within processor 904.

Computer system 900 may also include an input device 932. In one example, a user of computer system 900 may enter commands and/or other information into computer system 900 via input device 932. Examples of an input device 932 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 932 may be interfaced to bus 912 via any of a variety of interfaces (not shown) including, but not limited to, a serial interface, a parallel interface, a game port, a USB interface, a FIREWIRE interface, a direct interface to bus 912, and any combinations thereof. Input device 932 may include a touch screen interface that may be a part of or separate from display 936, discussed further below. Input device 932 may be utilized as a user selection device for selecting one or more graphical representations in a graphical interface as described above.

A user may also input commands and/or other information to computer system 900 via storage device 924 (e.g., a removable disk drive, a flash drive, etc.) and/or network interface device 940. A network interface device, such as network interface device 940, may be utilized for connecting computer system 900 to one or more of a variety of networks, such as network 944, and one or more remote devices 948 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 944, may employ a wired and/or a wireless mode of communication. In general, any network topology may be used. Information (e.g., data, software 920, etc.) may be communicated to and/or from computer system 900 via network interface device 940.

Computer system 900 may further include a video display adapter 952 for communicating a displayable image to a display device, such as display 936. Examples of a display device include, but are not limited to, a liquid crystal display (LCD), a cathode ray tube (CRT), a plasma display, a light emitting diode (LED) display, and any combinations thereof. Display adapter 952 and display 936 may be utilized in combination with processor 904 to provide graphical representations of aspects of the present disclosure. In addition to a display device, computer system 900 may include one or more other peripheral output devices including, but not limited to, an audio speaker, a printer, and any combinations thereof. Such peripheral output devices may be connected to bus 912 via a peripheral interface 956. Examples of a peripheral interface include, but are not limited to, a serial port, a USB connection, a FIREWIRE connection, a parallel connection, and any combinations thereof.

Further referring to FIG. 9, a computing device may include any computing device as described in this disclosure, including without limitation a microcontroller, microprocessor, digital signal processor (DSP) and/or system on a chip (SoC) as described in this disclosure. A computing device may include, be included in, and/or communicate with a mobile device such as a mobile telephone or smartphone. A computing device may include a single device having components as described above operating independently, or may include two or more such devices and/or components thereof operating in concert, in parallel, sequentially or the like; two or more devices, processors, memory elements, and the like may be included together in a single computing device or in two or more computing devices. A computing device may interface or communicate with one or more additional devices as described below in further detail via a network interface device.

In some embodiments, and still referring to FIG. 9, a computing device may be a component of a combination of at least a computing device; at least a computing device may include, as a non-limiting example, a first computing device or cluster of computing devices in a first location and a second computing device or cluster of computing devices in a second location. At least a computing device may include one or more computing devices dedicated to data storage, security, distribution of traffic for load balancing, and the like. At least a computing device may distribute one or more computing tasks as described below across a plurality of computing devices of computing device, which may operate in parallel, in series, redundantly, or in any other manner used for distribution of tasks or memory between computing devices. At least a computing device may be implemented, as a non-limiting example, using a “shared nothing” architecture.

With continued reference to FIG. 9, one or more programs or software instructions may include a principal program and/or operating system; principal program and/or operating system may be a program that runs automatically upon startup of a computing device and manages computer hardware and software resources. Principal program and/or operating system may include “startup,” “loop,” and/or “main” programs on a microcontroller; such programs may initialize hardware resources and subsequently iterate through a series of instructions to make function calls, read in data at input ports, output data at output ports, and process interrupts caused by asynchronous data inputs or the like. Principal program and/or operating system may include, without limitation, an operating system, which may schedule program tasks to be implemented by one or more processors, act as an intermediary between one or more programs and inputs, outputs, hardware, and/or memory. Examples of operating systems include without limitation Unix, Linux, Microsoft Windows, Android, Disc Operating System (DOS) and the like. Operating systems may include, without limitation, multi-computer operating systems that run across multiple computing devices, real-time operating systems, and hypervisors. A “hypervisor,” as used in this disclosure, is an operating system that runs a virtual machine and/or container, where virtual machines and/or containers create virtual interfaces for programs that mimic the behavior of hardware elements such as processors and/or memory; interactions with such virtual interfaces appear, to programs executed on virtual machines, to function as interactions with physical hardware, while in reality the hypervisor and/or programs such as containers (1) receive inputs from programs to the virtual resources and allocate such inputs to physical hardware that is not directly accessible to the programs, and (2) receive outputs from physical hardware and transmit such outputs to the programs in the form of apparent outputs from the virtual hardware. In some cases, one or more of computing system 900, processor 904, and memory 908 may be virtualized; that is, a virtual machine and/or container may interact directly with such computing system 900, processor 904, and/or memory 908, while managing communications therefrom and thereto via a virtual interface with programs. Computer virtualization may include dividing, or augmenting computing resources into a virtual machine, operating system, processor, and/or container. Virtualization of computer resources may be implemented through use of (1) multiple components, or portions thereof, working in concert, as if they were one unified (virtual) component; and/or (2) a portion of one or more components working as though it were a complete (virtual) component. For instance, where processor 904 comprises a plurality of processors and/or processor cores, virtualization may, in some cases, simulate or emulate a single (virtual) processor whose functions are allocated to one or more of the plurality of processors and/or processor cores. In this case, while processor 904 may be said to be virtualized, the processor 904, nevertheless, comprises actual hardware processor(s) or portion(s) thereof. Accordingly, in this disclosure, where a processor is said to perform instructions, such processor may comprise a virtualized processor, comprising a plurality or portion of hardware processors. Likewise, in this disclosure, where a memory is said to contain (i.e., store) instructions, such memory may comprise a virtualized memory, comprising a plurality or portion of memories. Technologies that enable such virtualization include (1) QEMU, www.qemu.org; (2) VMware by Broadcom Inc of Palo Alto, California; (3) VirtualBox by Oracle Corporation headquartered in Austin, Texas; and (4) kernel-based virtual machine (KVM) www.linux-kvm.org.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A device for multiplexed optical biosensing, wherein the device comprises:
at least an optical waveguide, each optical waveguide of the at least an optical waveguide having an input and an output;
at least a light source optically coupled to the input of each optical waveguide, wherein the at least a light source is configured to transmit an input signal into the optical waveguide;
a plurality of optical signal modulators optically coupled to each optical waveguide, wherein:
each optical signal modulator of the plurality of optical signal modulators is fluidically connected to a fluidic channel containing at least a biological analyte;
the plurality of optical signal modulators is configured to produce a plurality of modified signals, wherein each modified signal of the plurality of modified signals is generated by an optical signal modulator of the plurality of optical signal modulators by modifying the input signal based on the at least a biological analyte, and each modified signal of the plurality of modified signals has a distinct optical characteristic from all other modified signals of the plurality of modified signals; and
a receiver module optically coupled to the output of each optical waveguide, the receiver module further comprising:
at least a light sensor optically coupled to the output of the optical waveguide and configured to detect the plurality of modified signals; and
circuitry configured to record the plurality of detected modified signals, wherein the receiver module is configured to detect each modified signal of the plurality of modified signals based on its distinct optical characteristic and determine presence of each of the at least a biological analyte based on a respective detected modified signal.

2. The device of claim 1, wherein the plurality of optical signal modulators further comprises:
a first optical signal modulator configured to generate a first modified signal type; and
a second optical signal modulator configured to generate a second modified signal type, wherein the first modified signal type is distinct from the second modified signal type.

3. The device of claim 2, wherein:
the first optical signal modulator is an interferometer; and
the second optical signal modulator is a resonator.

4. The device of claim 1, wherein the distinct optical characteristic further comprises a distinct frequency transmission spectrum.

5. The device of claim 4, wherein the receiver module is configured to detect each modified signal using the distinct frequency transmission spectrum by analyzing a frequency spectrum of an output from the output end of the waveguide.

6. The device of claim 5, wherein:
the output has a temporal frequency continuum; and
the receiver module is configured to analyze the frequency spectrum as a function of the temporal frequency continuum.

7. The device of claim 6, wherein the receiver module analyzes the frequency spectrum based on stored temporal data of the temporal frequency continuum.

8. The device of claim 6, wherein the receiver module analyses the frequency spectrum using an optical frequency discriminator.

9. The device of claim 8 wherein the optical frequency discriminator includes an interferometer.

10. The device of claim 8 wherein the optical frequency discriminator includes a grating.

11. The device of claim 5, wherein:
the output has a simultaneous frequency continuum; and
the receiver module further comprises an optical frequency discriminator.

12. The device of claim 11, wherein the optical frequency discriminator includes an interferometer.

13. The device of claim 11, wherein the optical frequency discriminator includes a grating.

14. The device of claim 5, wherein each modified signal of the plurality of modified signals includes at least one frequency domain extremum that does not overlap with an extremum of any other modified signal of the plurality of modified signals.

15. The device of claim 14, wherein the receiver module is configured to analyze the frequency spectrum of the output by mapping extrema to optical modulators.

16. The device of claim 15, wherein the receiver module performs the mapping using a clustering algorithm.

17. The device of claim 5, wherein the receiver module is configured to analyze the frequency spectrum of the output using a Fourier transform.

18. The device of claim 1, wherein:
the device includes a plurality of fluidics channels; and
the plurality of fluidics channels includes a modulator-specific channel for each optical modulator of the plurality of optical modulators.

19. The device of claim 1, wherein the plurality of optical modulators includes at least four optical modulators.

20. The device of claim 1, wherein the at least an optical waveguide further comprises a plurality of optical waveguides.
